(19) United States
(12) Patent Application Publication
(10) Pub. No.: US 2026/0165578 A1
(43) Pub. Date: Jun. 18, 2026

(54) HARDWARE-AGNOSTIC PHOROPTER CONTROL TECHNIQUES FOR VISION EXAMINATIONS

(71) Applicant: DigitalOptometrics LLC, Lake Success, NY (US)

(72) Inventors: William Kenneth Van Cleave, Abilene, TX (US); Kurt Schaeffer, Lake Success, NY (US); Robert Hansel, Lake Success, NY (US); Paul E. Muehlhausen, Lake Success, NY (US); Mobin Varghese, Lake Success, NY (US); Charles A. Dowalo, Lake Success, NY (US); Howard S. Fried, Lake Success, NY (US); Alex Louw, Lake Success, NY (US); Douglas C. Viney, Lake Success, NY (US)

(21) Appl. No.: 19/417,785

(22) Filed: Dec. 12, 2025

Related U.S. Application Data
(60) Provisional application No. 63/733,803, filed on Dec. 13, 2024.

(51) Int. Cl. A61B 3/032; A61B 3/00; G16H 40/67; H04L 67/125

(52) U.S. Cl. CPC A61B 3/032; A61B 3/0025; A61B 3/0033; G16H 40/67; H04L 67/125

(57) ABSTRACT

Systems and techniques are disclosed for supporting multiple deployment configurations to accommodate a wide range of clinical and operational environments. In some implementations, data indicating an instruction to perform a phoropter operation for a vision examination is obtained. A first command corresponding to the phoropter operation is determined. A particular plugin corresponding to the first command is selected from a library of plugins and based on the classification. The library identifies (i) a set of phoropter classifications for the vision examination and (ii) one or more hardware-specific command rules for each phoropter classification included in the set of phoropter classifications. A second command for the phoropter is generated by applying one or more particular hardware-specific command rules specified by the particular plugin. The second command is provided for output in response to the instruction.

### CROSS-REFERENCE TO RELATED APPLICATIONS

This application claims the benefit of U.S. Provisional Application No. 63/733,803, filed on Dec. 13, 2024, the contents of which are incorporated by reference in its entirety.

### TECHNICAL FIELD

This disclosure describes technology generally relating to hardware configurations of optometric equipment, and more specifically, phoropter control techniques.

### BACKGROUND

Examples of eye-focused healthcare industries include optometry, ophthalmology, tele-optometry, and optical retail and services. These industries involve a vision examination (e.g., eye examination), which is a comprehensive evaluation of a person's eyesight and overall eye health. A vision examination is typically conducted by an optometrist, ophthalmologist, or a refractionist. The vision examination may involve a series of tests to assess visual acuity (sharpness of vision), determine the need for corrective lenses (such as glasses or contact lenses), and check for common eye conditions such as astigmatism, nearsightedness (myopia), farsightedness (hyperopia), or presbyopia. A vision examination may include assessing eye movement, coordination, depth perception, peripheral vision, and the health of the internal and external structures of the eye (e.g., retina, cornea, and optic nerve). Vision examinations are conducted using optometric equipment, such as a phoropter, autorefractor, or retinoscope, which measure refractive errors and establish corrective prescriptions. Dilation or imaging techniques may also be employed to examine the health of the eye in more detail.

Vision examinations may involve a subjective refraction, which is a technique to determine the combination of lenses that will provide the best corrected visual acuity (BCVA). A subjective refraction examination is a clinical examination used by orthoptists, optometrists, and ophthalmologists to determine a user's need for refractive correction in the form of glasses or contact lenses.

### SUMMARY

This disclosure describes systems and techniques for applying a compatibility engine for improving the integration and use of optometric equipment during the administration of vision examinations. For example, the systems disclosed herein enable the administration of refraction and vision assessments that may involve the use of phoropters manufactured by different providers. In some implementations, a modular software platform enables seamless control of diverse phoropter and eye chart hardware from various manufacturers, facilitated by vendor-specific plugins and a standardized graphical user interface. Various implementations support different vision examination formats, including the location of administration (local, remote) and the level of workflow automation (manual, semi-automated, fully-automated). The compatibility engine also addresses the challenges of interoperability, accessibility, and accuracy in vision care, further enabling versatile and scalable solutions for both clinical and remote eye exam settings.

The systems and methods described herein provide technical solutions to hardware interoperability issues implicated in the remote and automated control of medical diagnostic equipment, and specifically to the real-time administration of vision examinations. These solutions involve a hardware abstraction layer, which improves the functioning of computing systems by enabling a single, universal software platform to control a heterogeneous ecosystem of optometric devices. As discussed herein, a client device or server is configured to act as a universal translator and thereby establishes a standardized control interface between a high-level clinical workflow application and a plurality of disparate, vendor-specific hardware components.

Further, conflict may arise from the operational requirements of remote examination systems when applied in clinical environments with diverse hardware. The field of optometry relies on specialized hardware, such as phoropters and eye charts, from many different manufacturers. This equipment often has disparate configurations and proprietary command languages, with each manufacturer's device using its own unique set of commands and communication protocols to function. This lack of a common communication standard creates a significant technical barrier to the development and deployment of scalable, remote, and automated examination platforms.

This hardware fragmentation creates a technical paradox for system developers and creates a technical trade-off that results in system failure or inefficiency. A conventional “device-specific” approach, which involves developing a separate, bespoke software application for each type of hardware, results in software fragmentation. This approach is not scalable, requires operators to learn multiple interfaces, and prevents the implementation of standardized clinical workflows across different sites, thereby defeating the purpose of a centralized system. Conversely, an approach that ignores these hardware differences and attempts to use a single, generic command set would simply fail to operate most equipment, making such a system technically infeasible for deployment in any real-world clinical setting. Therefore, a specific, unmet need exists for computer-implemented systems that can resolve this conflict by intelligently and dynamically translating universal, hardware-independent commands into the specific, proprietary formats required by a diverse range of optometric devices.

The systems and techniques described herein address these and other unmet need through use of a centralized compatibility engine configured to receive high-level, hardware-independent commands representing clinical actions. The engine performs a specific, dynamic translation process by selecting, from a library of plugins, a particular plugin corresponding to the classification of the connected hardware. Each plugin contains the hardware-specific command rules for a particular device classification. The engine applies these rules to generate a second, hardware-specific command that is in a format executable by the target device. This process represents a specific improvement to computer functioning and interoperability, as it allows the system to leverage a single, standardized control interface to operate a wide array of otherwise incompatible hardware, thereby overcoming a fundamental technical barrier in the field of remote medical diagnostics.

The systems described herein also support multiple deployment configurations to accommodate a wide range of clinical and operational environments. For example, a control panel and compatibility engine may be distributed across separate computing systems connected via a network, such as a cloud-hosted connector service, or co-located on a single device to operate in a standalone, offline configuration. This flexibility enables deployment in settings with constrained infrastructure or limited network connectivity, while still supporting the same standardized workflows and plugin-based hardware abstraction. The connector service further allows for secure relay of commands, real-time updates, and optional integration with other infrastructure services such as software update delivery and monitoring.

The software platform includes a layered plugin architecture that enhances the system's extensibility and maintainability. Each plugin is associated with a specific functional layer (e.g., phoropter control, eye chart presentation, user interface layout) and is responsible for translating high-level commands into the corresponding device-specific protocol. For example, engine plugins may be configured to generate command sets for controlling phoropter hardware, while control panel plugins define user interface layouts that simulate familiar vendor-specific designs. This modularity allows the systems disclosed herein to be tailored to the specific hardware present at the examination site and enables future updates to individual components without requiring changes to the core platform.

In some implementations, a system includes program plugins that provide structured guidance to the remote technician or doctor during the execution of a refraction or other clinical test. These program plugins define sequential workflows that ensure examinations are conducted in a consistent and clinically validated manner, regardless of operator experience or hardware configuration. Certain program plugins may include optional feedback modules, enabling integration with patient-facing features such as voice interaction or speech recognition. This facilitates real-time capture of verbal responses, which may be used to guide examination progression or confirm patient understanding.

Additionally, graphical user interfaces rendered on the control panel may be dynamically adjustable and repositioned or resized to accommodate the technician's preferences or screen setup. Such interfaces provide a standardized set of controls for interacting with the optometric equipment, regardless of manufacturer. By emulating familiar control schemes and embedding programmatic examination logic, the system may minimize training burden and promotes adoption across clinical networks.

The systems and techniques disclosed herein further allow for interchangeable combinations of supported optometric hardware. For example, different phoropter types and eye chart systems may be used in conjunction with one another under a unified control platform. A compatibility engine ensures that each device receives properly formatted instructions derived from a shared clinical workflow, thereby enabling composable, vendor-agnostic examination lanes. This capability is particularly valuable in environments where hardware upgrades or replacements occur over time or across different sites, as it allows new devices to be integrated without disrupting the overarching examination logic or interface consistency.

In one general aspect, a method may be implemented by one or more computing devices. The method includes obtaining data indicating an instruction to perform a phoropter operation for a vision examination. The instruction specifies a classification for a phoropter for performing the phoropter operation. The method also includes determining a first command corresponding to the phoropter operation. Further, the method includes selecting, from a library of plugins and based on the classification, a particular plugin corresponding to the first command. The library identifies (i) a set of phoropter classifications for the vision examination and (ii) one or more hardware-specific command rules for each phoropter classification included in the set of phoropter classifications. The method also includes generating, by applying one or more particular hardware-specific command rules specified by the particular plugin, a second command for the phoropter. The second command is in a format that is executable by the phoropter. The second command is provided for output in response to the instruction.

One or more implementations include the following optional features. For example, in some implementations, the first command is in a hardware-independent format. In such implementations, the first command is based on identifying a specific clinical step to be performed during the vision examination.

In some implementations, the second command is in a hardware-specific format. In such implementations, the second command is determined based on a command language specification associated with the classification for the phoropter.

In some implementations, generating the second command includes generating a command to adjust a hardware component of the phoropter.

In some implementations, the instruction is obtained from a graphical user interface (GUI) executing on the computing device.

In some implementations, the method further includes establishing a network connection between the one or more computing devices and the computing device from which the data indicating the instruction is obtained.

In some implementations, the method further includes receiving, from the phoropter, data indicating a connection status.

In some implementations, the library of plugins further includes a plurality of eye chart plugins. In such implementations, the method further includes obtaining data indicating an instruction to perform an eye chart operation. The method also includes determining a first eye chart command corresponding to the eye chart operation, and selecting, from the plurality of eye chart plugins, a particular eye chart plugin corresponding to a classification of an eye chart. Further, a second eye chart command is generated by applying a hardware-specific command rule from the particular eye chart plugin. The method also includes providing the second eye chart command for output to the eye chart.

In some implementations, the set of phoropter classifications includes a first classification specified by the instruction and a second classification different from the first classification. In such implementations, the method further includes obtaining second data indicating a second instruction to perform a second phoropter operation. The second instruction specifies the second classification for a second phoropter. The method also includes determining a third command corresponding to the second phoropter operation and selecting, from the library of plugins and based on the second classification, a second particular plugin corresponding to the third command. A fourth command for the second phoropter is generated by applying one or more hardware-specific command rules specified by the second particular plugin. The fourth command is in a format that is executable by the second phoropter. The method also includes providing the fourth command for output in response to the second instruction.

The details of one or more implementations of the disclosure are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram of an exemplary system for administering vision examinations using a compatibility engine.

FIG. 2 is a conceptual diagram of a technique for using hardware-agnostic command data in administering a vision examination.

FIG. 3 is a conceptual diagram of a technique for using a compatibility engine for generating hardware-agnostic command data for multiple phoropters.

FIG. 4A-4B are conceptual diagrams of exemplary interfaces for administering vision examinations using a compatibility engine.

FIG. 5 is a conceptual diagram of an exemplary interface for an eye chart plugin for use

FIG. 6 is a flow chart illustrating an exemplary process for controlling optometric equipment.

FIG. 7 is a flow chart illustrating an exemplary process of controlling a phoropter using a compatibility engine during a vision examination.

FIG. 8 is a flow chart illustrating an exemplary process of controlling a phoropter using a compatibility engine.

In the drawings, like reference numbers represent corresponding parts throughout.

### DETAILED DESCRIPTION

This disclosure describes systems and methods for enabling universal control of optometric equipment used in vision examinations, which may be administered in local, remote, or distributed environments. In general, the systems utilize a client device with a compatibility engine to orchestrate the examination process by translating commands received from a provider's control application into instructions executable by a plurality of disparate hardware devices. The system is configured to receive and process high-level, hardware-independent instructions from a standardized graphical user interface, which may be operated by a local technician, a remote provider, or an automated examination protocol.

As discussed below, system operation involves a multi-stage translation process where the compatibility engine obtains a first, universal command to adjust optometric equipment, determines the classification of the connected hardware, and selects a corresponding, hardware-specific plugin from a library. By applying the command language specifications and rules defined within the selected plugin, the engine generates a second, hardware-specific command that is in a format executable by the target device. This flexible, modular architecture enables a single, consistent software interface to control a heterogeneous ecosystem of phoropters and eye charts from various manufacturers. In some examples, the system enables direct, manual control by a human operator. In other examples, the system enables the execution of complex, automated clinical workflows. These examples improve the efficiency, scalability, and accuracy of vision care by overcoming the technical barriers of hardware interoperability.

As described herein, the term “module” generally refers to a component, unit, or logical grouping of functionality within a larger system. A module may perform a specific function or a set of related functions through software, hardware, firmware, or any combination. In the context of this specification, a module represents a logical subdivision of a larger component, such as an engine. For example, a compatibility engine may include a workflow logic module for applying clinical rules and a hardware abstraction module for translating commands. Functionality described as being performed by a particular module may be distributed across multiple software or hardware components. Conversely, functionality described as being performed by multiple distinct modules in the disclosure may be integrated into a single, combined component in some implementations. A system, subsystem, or engine may be composed of one or more modules.

As described herein, the term “engine” generally refers to one or more software modules, components, or services configured to perform a specialized data processing and transformation task. In the context of this specification, one function of the engine is to receive an input command in a first, universal format and generate a corresponding output command in a second, hardware-specific format. This transformation may be performed by applying a set of rules, specifications, or protocols that define the command language and communication requirements of the target hardware. The rules and specifications may be implemented as a library of modular, interchangeable plugins, which allows the engine to be dynamically configured to support a plurality of different hardware devices without altering its core logic. For example, the engine acts as an abstraction layer that decouples controlling software (such as the provider's remote application) from the specific (in some instances, proprietary) command languages of the various controlled hardware devices (such as phoropters and eye charts).

As described herein, the term “plugin” generally refers to a self-contained, interchangeable software component that may be configured to provide a specific set of data, rules, instructions, or functionality to a host application, such as the compatibility engine. A plugin allows for the separation of core engine logic from specific implementation details, thereby enabling modularity, flexibility, and extensibility. In the context of this specification, plugins are accessed by a compatibility engine to enable it to perform its workflow validation and command translation functions. For example, a plugin may be implemented to specify hardware-specific command languages and communication protocols for a particular model or vendor of optometric equipment (e.g., Phoropter Plugins, Eye Chart Plugins). As another example, a plugin may be implemented to specify a sequence of steps and conditional logic for a guided clinical workflow to be administered to a patient (e.g., Program Plugins). In other examples, a plugin specifies a configuration for a graphical user interface, such as the layout, appearance, and function of its controls (e.g., Control Panel Plugins).

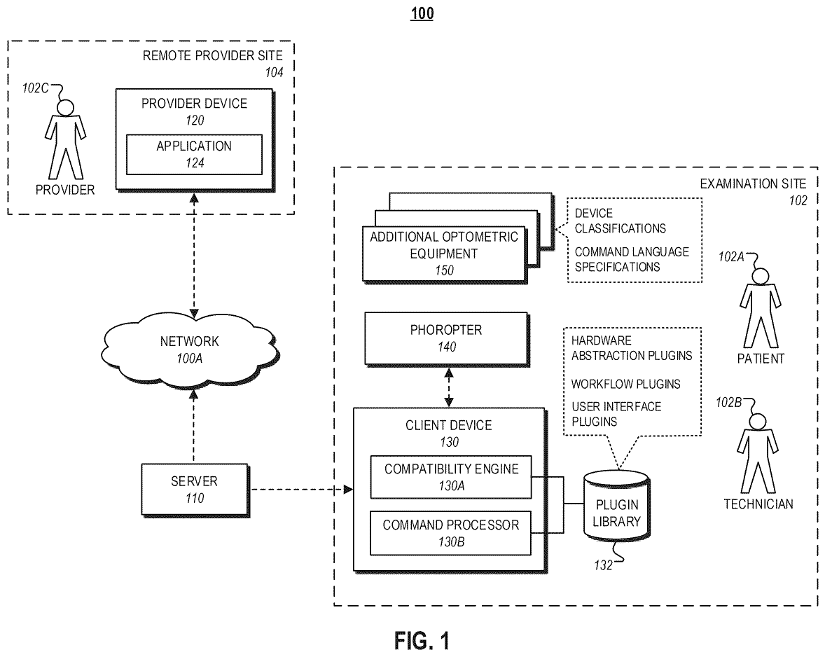

FIG. 1 illustrates an example of a system 100 for using a compatibility engine 130A to conduct a vision examination. As discussed below, the compatibility engine 130A enables the system 100 to convert generic commands from an equipment controller to hardware-specific commands, thereby enabling hardware-agnostic administration of vision examinations using the plurality of optometric equipment. In various implementations, the system 100 may be used to interface optometric equipment (e.g., phoropters) and eye charts with local or remote controllers.

In general, system 100 improves the control of additional optometric equipment 150 (and associated eye charts) by enabling compatibility with devices from multiple manufacturers through a modular and adaptable design. Various implementations of the system 100 may leverage cloud-based communication protocols (e.g., Azure Pub-Sub) to ensure seamless interaction between remote computing devices. This architecture enables scalability, adaptability, and efficient integration of hardware, thereby providing a standardized, flexible solution for remote and automated vision examinations.

In the example shown in FIG. 1, system 100 enables the administration of a vision examination involving a patient 102A, a technician 102B, and a provider 102C in relation to an examination site 102. In this example, the provider 102C is located in a provider site 104, which is remote from the examination site 102 (where patient 102A and local technician 102B are located). Provider 102C interacts with system 100 over a network 100A using a provider device 120, which further includes an application 122. The examination site 102 includes a client device 130, which interacts with system 100 over network 100A. The client device 130 is configured to communicate with a plurality of optometric equipment located in the examination site 102, including phoropter 140 (used to administer vision examination) and additional optometric equipment 150.

The vision examination shown in FIG. 1 is an example of an examination that is administered through use of local controller software running on client device 130, and conducted remotely by provider 102C through use of the application 122 on provider device 120. In this example, the client device 130 converts generic input commands into manufacturer-specific instructions for controlling phoropter 140 through use of controller software that includes the compatibility engine 130A and the command processor 130B. The local technician 102B manually operates the controller software on the client device 130 through, for example, a graphical user interface presented on a display associated with client device 130.

Provider 102C is trained to operate phoropter 140 and remotely conducts the vision examination through use of provider device 120. The provider 102C may be an individual trained to operate the phoropter 140, such as a medical doctor (e.g., ophthalmologist), an optometrist specialized in vision examinations, or an optician. Local technician 102B may be a healthcare professional that assists the provider 102C in administering a vision examination, such as an optician. While not specifically trained to operate the phoropter 140, local technician 102B may focus on fitting, dispensing, and adjusting eyewear based on the prescriptions provided by these eye care professionals.

Data communications between computing devices located in each of the provider site 104 and the examination site 102 are enabled through the network 100A. For example, the network 100A enables the provider 102C, using the provider device 120, to communicate with the patient 102A. The network 100A also enables communication between the provider device 120 and the client device 130, thereby facilitating remote control of the phoropter 140 by the provider 102C during the vision examination 102.

The server 110 functions as the central orchestrator for the system 100 and is configured to provide centralized services for supporting the functionality of the system 100. The server 110 also provides infrastructure support for an application (e.g., application 122) that executes on the client device 124. As illustrated, the server 110 may include logical modules for managing the core examination logic in managing the examination process between the provider device 120 and client device 130. The server 110 may be configured in various ways, including as a single physical server, a distributed cluster of virtual machines, or as a set of services deployed within a public or private cloud environment, for instance, using a microservices architecture.

To provide the functionality described herein, the server 110 interacts with several key components, including inputs from the provider device 120 and the client device 130. The server 110 may also interact with various data sources that store different types of data, such as health data, user data, and/or training data. This data includes historical and contextual information used by the server 110 to personalize and intelligently guide the vision examination. The data sources generally serve as the system's long-term memory, providing the system 100 with data needed to build a comprehensive snapshot of the patient and clinical situations.

The system 100 also includes a variety of user-facing and equipment devices. The provider device 120 is a computing device through which a remote provider 102C administers, supervises, and reviews a vision examination from a remote site 104. The provider device 120 enables the remote provider 102C to transmit high-level clinical commands to the server 110 and/or the client device 130 and to receive real-time data representing the state and results of the examination. The provider device 120 may be, for example, a desktop computer, a laptop computer, a tablet, or a smartphone

Similarly, a remote technician device (not shown in FIG. 1) is a computing device through which a remote technician administers or assists in the operation of the vision examination from a remote site. The remote technician device enables the remote technician to transmit operational or procedural instructions to the server 110 and to receive real-time data. This device may also be a desktop computer, a laptop computer, a tablet, or a smartphone.

Client device 130 facilitates the administration of the vision examination 102. This is accomplished using controller software, including the compatibility engine 130A and the command processor 130B. In the example shown in FIG. 1, client device 130 is physically located in the examination site 102 and used by the local technician 102B in the administration of vision examination 102. As discussed below, functionality provided by the compatibility engine 130A and the command processor 130B enables the ability to control and operate optometric equipment (e.g., phoropter 140, additional optometric equipment 150) in a hardware-agnostic fashion.

Specifically, during the vision examination 102, the compatibility engine 130A receives data from the phoropter 140. The received data includes information specifying a classification of the equipment, and a command language specification. For example, the information may include the name of the manufacturer, model number, and other identifying information about the phoropter 140. The command language specification is a language in which commands are transmitted to a controller (e.g., client device 130 or provider device 120), which may vary by manufacturer and model of the phoropter 140.

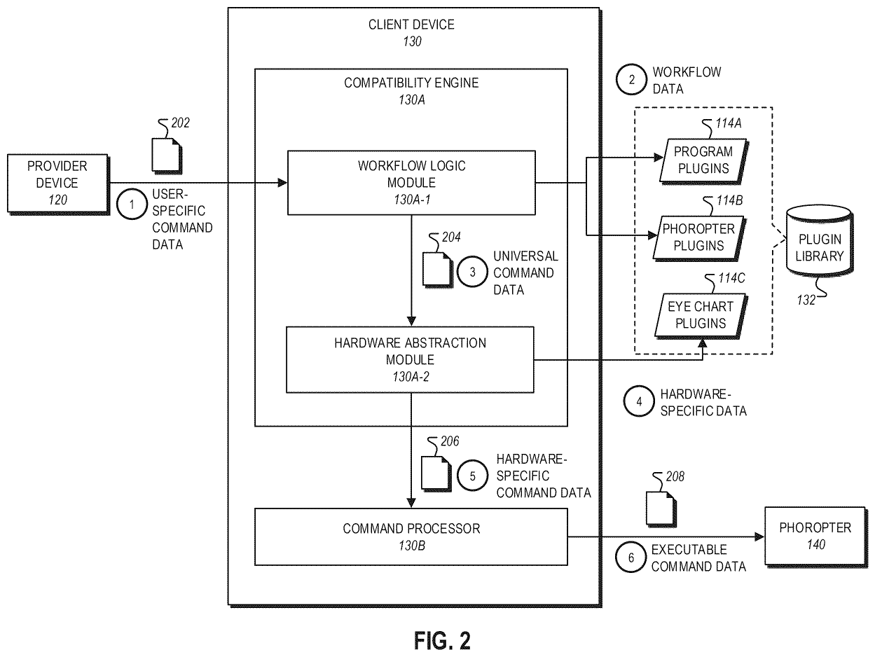

The compatibility engine 130A receives input data in a given command language from the local technician 102B. For example, as shown in FIG. 2, a graphical user interface (e.g., GUI 400) includes a plurality of icons that correspond to instructions corresponding to inputs by the provider 102C and for transmission to phoropter 140. In this example, the application 122 (running on the provider device 120) converts input received through a graphical user interface presented on the provider device 120 to computer-implemented instructions, which are then transmitted to the compatibility engine 130A. The instructions are then further processed by the command processor 130B prior to transmission to the phoropter 140.

The compatibility engine 130A also receives data indicating a classification of the phoropter 140, including data indicating a command language of the phoropter 140. The compatibility engine 130A determines whether the instruction received from the local technician 102B is compatible with the command language of the phoropter 140. Where the instructions and command language are compatible, the compatibility engine transmits the data to the command processor 130B. Thus, when instructions are compatible with the command language, the instructions may be forwarded to the command processor 130B, and then to a controller of the phoropter 140, where the instructions are performed.

The classification of the phoropter 140 may also include a classification of an eye chart used for administering the vision examination for a plurality of eye charts from different manufacturers or different models, the compatibility engine 130A, using the eye chart classification, determines a command language of the eye chart. The client device 130, using the eye chart command language, determines a common chart command for the eye chart. The common chart command is configured by the command processor 130B and the compatibility engine 130A to be output in a format that is compatible with the eye chart command language.

Where the compatibility engine 130A determines that instructions are not compatible with the command language of the phoropter 140, the compatibility engine 130A transmits the instructions to the command processor 130B along with additional instructions commanding the command processor 130B to convert the instructions to a second command. The second command is compatible with the command language of the phoropter 140. In some implementations, the second command may be a same language as the original command language of the phoropter 140.

Because there are several manufacturers and models of phoropter 140, the compatibility engine 130A is configured to specify command language specifications for the additional optometric equipment 150 of diverse types. In addition, the application 122 generates the GUI 400 with a consistent interface regardless of a classification or model of the phoropter 140. Thus, the provider 102C is allowed to control additional optometric equipment 150 without the need to understand the intricacies of each individual equipment among the additional optometric equipment 150. For example, the additional optometric equipment 150 may be a plurality of phoropters of diverse types.

A plugin library 132 may be installed by the local technician 102B or the provider 102C. In some implementations, some aspects of the plugin library 132 may be installed and implemented in the compatibility engine 130A. The plugin library 132 enables changes to the application 122, compatibility engine 130A, and controllers for the client device 130 or phoropter 140 without need to fully install or update software, thus providing greater flexibility. The plugin library 132 may include one or more hub plugins that enable communications for sending commands from the client device 130 to the phoropter 140. For example, the plugin library 132 may include a connection engine (e.g., Azure Pub-Sub) that enables a network connection between the provider device 120 and the phoropter 140.

The plugin library 132 may include different plugin types that are each tailored to interface with different components of the system 100. These plugin types include, but are not limited to, hub plugins, hardware abstraction plugins, eye chart plugins, and workflow and user interface plugins. Each type provides a specific abstraction layer or control mechanism for bridging differences in hardware capabilities, communication protocols, or user interaction models between optometric devices from different manufacturers.

For example, hub plugins may be configured to manage communications between the client device 130 and the hardware controllers of optometric devices, such as phoropter 140. Hub plugins translate network-agnostic messages into connection-specific communication protocols. For example, a hub plugin may enable messaging over a local serial port, TCP/IP socket, or cloud-based messaging system such as Azure Pub-Sub. This allows the compatibility engine 130A to remain unaware of lower-level transport details while still ensuring timely and reliable message delivery to and from hardware.

As another example, hardware abstraction plugins may define the command syntax, operational rules, and device-specific mappings needed to issue low-level commands to optometric hardware, including phoropter 140 and additional optometric equipment 150. Hardware abstraction plugins encapsulate knowledge such as manufacturer-specific command languages, required timing between instructions, and supported feature sets. The compatibility engine 130A consults these plugins when converting a hardware-independent clinical instruction (e.g., “add +1.00 to right eye”) into a format compatible with the underlying device's protocol.

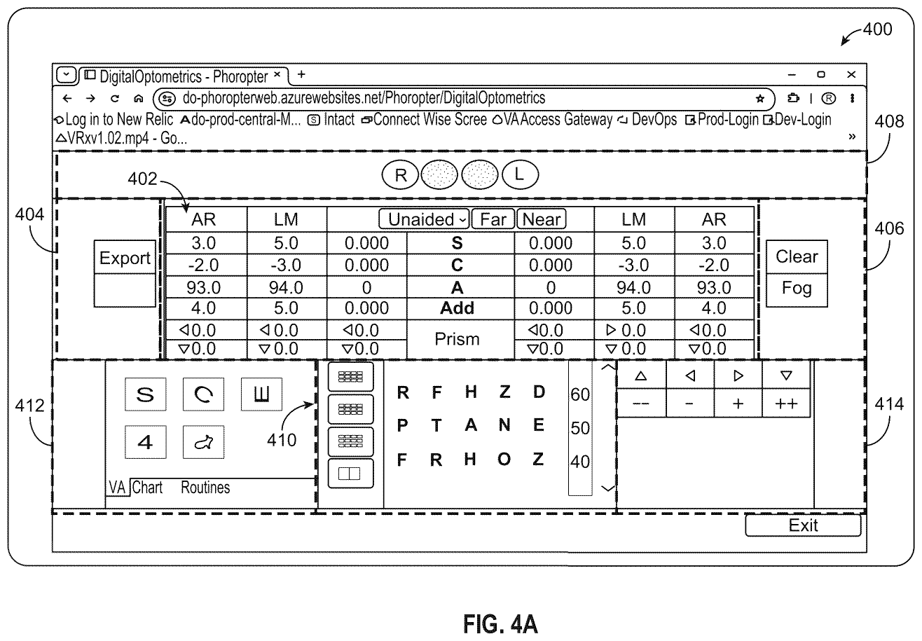

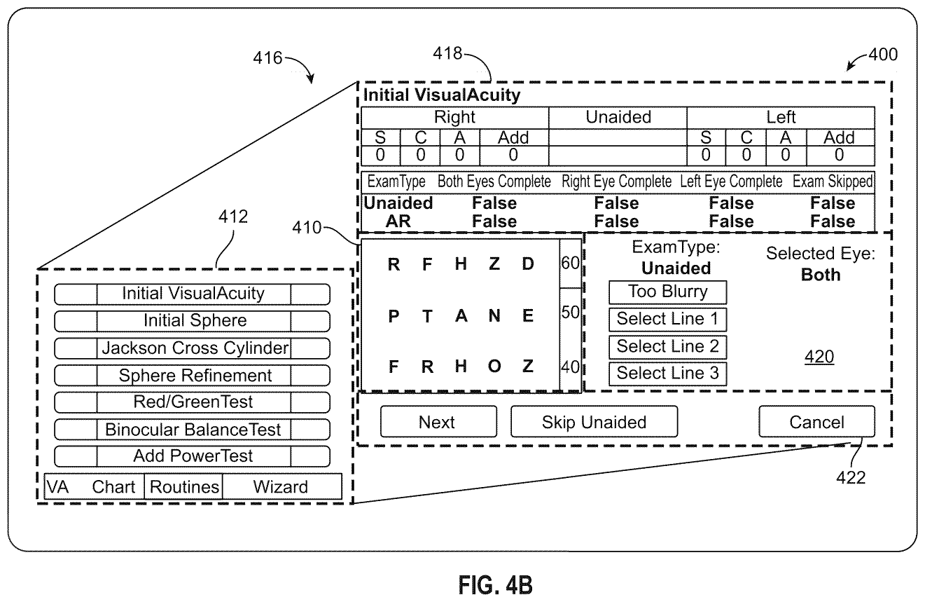

Eye chart plugins may be specialized hardware abstraction plugins designed to control external vision charts connected to or integrated with a phoropter. For instance, because eye charts may vary by resolution, masking ability, coordinate mapping, and control interface, each plugin encapsulates the operational parameters and control routines required to render charts on specific devices. These plugins allow a common interface (e.g., GUI 400 shown in FIG. 4A) to present a consistent chart control experience regardless of the specific chart hardware deployed at the examination site 102.

Further, workflow and user interface plugins facilitate the presentation of tailored graphical user interfaces and exam workflows depending on the clinical setting or equipment configuration. For example, the same software may provide an advanced configuration with full refraction tools when the provider 102C is operating the system, and a simplified UI with guided prompts when used by the technician 102B. These plugins enable dynamic adaptation of the client device 130 or provider device 120 interface based on user roles, patient status, or device capabilities.

The system 100 may be implemented to facilitate various types of vision examination formats in accordance with the principles discussed above. In this respect, in various implementations, hardware elements of the system 100 (e.g., provider device 120, client device 130, phoropter 140) may be arranged in different ways to support different vision examination formats ways using similar software functionality discussed above in reference to the example shown in FIG. 1. In some examples, the system 100 is configured to support a fully-remote vision examination in which each of the provider device 120, client device 130, and phoropter 140 are remote from one another (e.g., physically located in different locations). In such examples, client device 130 is implemented as a remote server that is configured to operate as a remote equipment controller. In this way, functionality of the compatibility engine 130A and the command processor 130B may be provided via the network 100A without necessitating the client device 130 to be physically co-located with the phoropter 140 (or otherwise without requiring the client device 130 to be physically located on the premises of the examination site 102).

In other examples, the system 100 is configured to support a fully-local vision examination in which each of the provider device 120, client device 130, and phoropter 140 are located in the same physical location (e.g., examination site 102). In such examples, network 100A may be a local area network (LAN) or some other suitable means of enabling data communications between provider device 120, client device 130, and phoropter 140. In such examples, client device 130 is implemented as a local computing device that is configured to operate as a local equipment controller. In this way, functionality of the compatibility engine 130A and the command processor 130B may be provided without reliance on a wide area network (WAN) or an Internet-based network.

FIG. 2 is a conceptual diagram illustrating a technique 200 for dynamically translating hardware-agnostic command data into hardware-specific instructions executable by phoropter 140 during the administration of a vision examination. The technique 200 demonstrates how a clinical instruction initiated by a provider device 120 is received, interpreted, and translated by a compatibility engine 130A within a client device 130 by leveraging a plugin library 132 to generate hardware-specific output. Technique 200 enables executable instructions to be transmitted to the phoropter 140 via a command processor 130B, which facilitates seamless, standardized control of heterogeneous optometric hardware.

As shown, the client device 130 includes two primary software components, including the compatibility engine 130A and the command processor 130B. The compatibility engine 130A orchestrates the interpretation of high-level, provider-originated examination logic and performs the translation of universal, hardware-independent command structures into device-specific instructions. The command processor 130B handles low-level formatting, packaging, and delivery of the final executable command data for application to the connected phoropter 140. This architectural division allows for modular implementation and testing, enhancing scalability and maintainability across examination environments.

In various implementations, the compatibility engine 130A includes a workflow logic module 130A-1 and a hardware abstraction module 130A-2. The workflow logic module 130A-1 functions as a high-level controller that sequences clinical operations based on preconfigured protocols and generates standardized command templates that abstract away hardware-specific constraints. The hardware abstraction module 130A-2 serves as the translation layer, converting abstract, hardware-independent commands into concrete instructions formatted according to the syntax and operational rules of the phoropter 140. This layered structure allows the system to support a wide range of devices without modifying upstream examination logic.

The translation process discussed above leverages a plugin library 132, which supplies reusable, modular rulesets describing both examination workflows and device-specific control logic. The plugin library 132 includes one or more program plugins 114A that define procedural sequences for particular clinical protocols (e.g., subjective refraction workflows, visual acuity tests, Jackson Cross Cylinder sequences). Additionally, the plugin library 132 includes phoropter plugins 114B and eye chart plugins 114C, which contain device-specific command rules, syntax mappings, and supported parameter sets for different makes and models of phoropters and chart displays. In some implementations, plugins are loaded into memory of the client device 130 during session initialization. In other implementations, plugins are retrieved by the client device 130 on-demand from a local cache or networked plugin service.

Technique 200 begins when a provider initiates a clinical command from the provider device 120. For example, the provider may request a Jackson Cross Cylinder (JCC) axis refinement operation. This request is encoded as user-specific command data 202, which is transmitted over the network to the client device 130. The user-specific command data 202 includes high-level metadata describing the requested action, the context of the examination, and any relevant parameters (e.g., cylinder power probe value or axis angle increment).

Upon receiving the user-specific command data 202, the workflow logic module 130A-1 retrieves the appropriate program plugin 114A from the plugin library 132. The selected plugin provides the procedural logic for the requested clinical operation, including any conditional rules, branching sequences, or pre/post conditions. Based on the plugin's encoded logic, the workflow logic module 130A-1 determines the first specific clinical step in the operation and generates universal command data 204. The universal command data 204 is structured in a hardware-agnostic format that represents the intended clinical effect (e.g., “rotate axis to 90 degrees,” “display optotype line 4,” “apply +0.25D sphere adjustment”) without requiring knowledge of the connected hardware's protocol.

The universal command data 204 is forwarded to the hardware abstraction module 130A-2. To execute the translation, the hardware abstraction module 130A-2 queries the plugin library 132 to identify and retrieve a phoropter plugin 114B corresponding to the classification of the connected phoropter 140. Each phoropter plugin encapsulates device-specific configuration logic, such as acceptable value ranges, byte-level command encodings, transmission timing constraints, and protocol syntax. Based on this logic, the hardware abstraction module 130A-2 synthesizes hardware-specific command data 206, which expresses the original universal command using the precise structure, semantics, and low-level format required by phoropter 140. For example, a generic instruction to “rotate axis to 90 degrees” may be converted into a hexadecimal packet conforming to a vendor's serial protocol specification.

The hardware-specific command data 206 is provided to the command processor 130B, which performs data preparation steps needed to convert the instruction into executable command data 208. These steps may include encoding commands for transmission over USB, serial, or wireless interfaces, applying checksum validation, and aligning with any required transport-layer framing. The command processor 130B transmits the executable command data 208 to the phoropter 140, which executes the command by adjusting its optical or mechanical components accordingly. This modular architecture enables real-time, deterministic control of clinical equipment regardless of manufacturer or protocol differences.

In some implementations, the plugin library 132 is hosted locally within the client device 130. In other configurations, particularly in cloud-connected examination environments, the plugin library 132 may be stored in a remote repository and accessed by the client device 130 over a secure network connection. In such scenarios, a caching strategy may be used to pre-load frequently accessed plugins to reduce latency. The system may also be configured to verify plugin integrity using cryptographic signatures, ensuring that only verified command templates are used in clinical operations.

The compatibility engine 130A may also be implemented as part of a containerized microservices architecture within the client device 130. Such implementations enable independent scaling of the workflow logic module 130A-1 and hardware abstraction module 130A-2. This allows the system to optimize resource usage depending on whether the examination site is operating in a fully automated, technician-supervised, or remote-only configuration. In fully automated workflows, the compatibility engine 130A may operate in a headless mode, receiving examination instructions from a central server and directly issuing control signals to phoropter 140 without user intervention.

As discussed herein, the flexible and extensible architecture of the system 100 supports a variety of deployment configurations while ensuring that clinical commands remain portable across heterogeneous hardware environments. By separating workflow logic from hardware implementation details, technique 200 improves scalability, reduces software fragmentation, and enables consistent, high-quality care delivery across diverse examination settings.

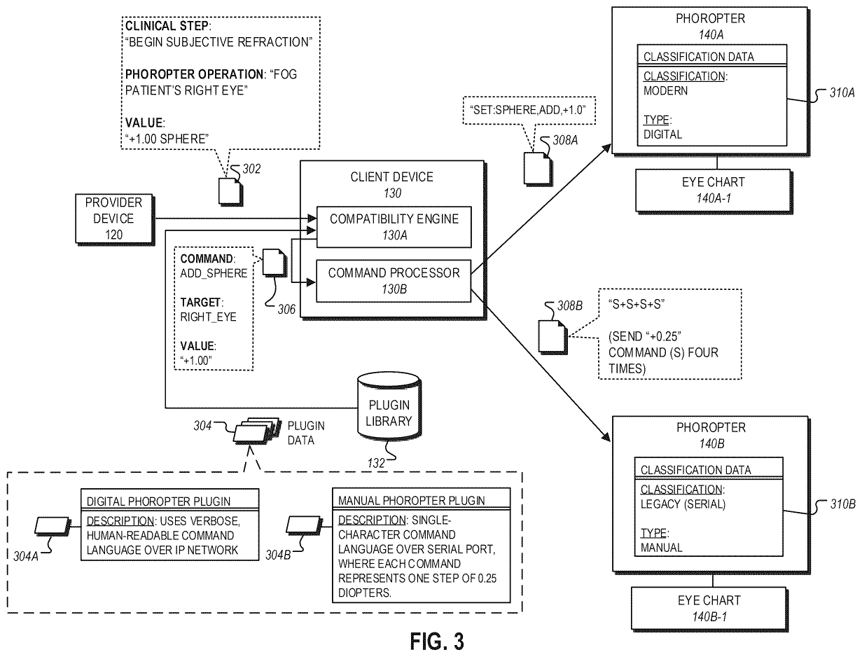

FIG. 3 is a conceptual diagram of a technique 300 for dynamically translating high-level, hardware-agnostic commands into hardware-specific executable instructions for phoropter operation. Technique 300 shows how a client device 130, via a compatibility engine 130A and a command processor 130B, enables the same clinical instruction to control either a modern digital phoropter 140A or a legacy manual phoropter 140B. In this example, despite each phoropter being associated with different command formats and communication protocols, they may be controlled during a vision examination using a shared software interface.

The workflow begins when a remote or local provider uses a provider device 120 to initiate a vision examination action. In the example shown in FIG. 3, the action is represented as clinical step data 302, which includes a high-level phoropter operation, such as “FOG PATIENT'S RIGHT EYE” as part of a subjective refraction workflow. The corresponding value, “+1.00 SPHERE,” specifies an intended lens adjustment for the patient. This instruction is transmitted to the client device 130 as a local execution node.

Upon receiving the clinical step data 302, the compatibility engine 130A interprets the instruction and generates universal command data 306. This data is expressed in a hardware-independent structure and includes a standardized command identifier (e.g., “ADD_SPHERE”), a target designation (e.g., “RIGHT_EYE”), and a numerical value (e.g., “+1.00”). This abstract representation allows clinical software to issue consistent instructions across a heterogeneous ecosystem of optometric hardware without embedding device-specific knowledge in the workflow logic.

The compatibility engine 130A accesses a plugin library 132 to translate the universal command into a device-specific format. The plugin library 132 includes a collection of modular plugins, such as a digital phoropter plugin 304A and a manual phoropter plugin 304B. Each element encapsulates, for instance, translation rules, command syntax, and communication mechanisms for a specific hardware classification. The compatibility engine 130A selects the appropriate plugin based on metadata associated with the connected phoropter, such as classification tags or interface properties.

In the first scenario, the connected optometric equipment is a modern phoropter 140A with classification data 310A identifying it as “MODERN” and “DIGITAL.” The compatibility engine 130A selects the digital phoropter plugin 304A, which specifies that the device communicates using a verbose, human-readable command language over an IP network. Applying the transformation rules in plugin data 304, the compatibility engine 130A converts the universal command data 306 into hardware-specific command data 308A, such as “SET:SPHERE, ADD, +1.0.” This command is then processed by the command processor 130B, which applies any required transport-layer encoding or framing to produce executable command data 310A. The final command is transmitted to phoropter 140A to perform the optical adjustment.

In the second scenario, the connected optometric equipment is a legacy phoropter 140B with classification data 310B identifying it as “LEGACY (SERIAL)” and “MANUAL.” In this scenario, the compatibility engine 130A selects the manual phoropter plugin 304B, which defines a compact, single-character command language communicated over a serial interface. In this protocol, each “S” command corresponds to a discrete +0.25 diopter increment. To achieve the desired +1.00 sphere change, the compatibility engine 130A generates four successive step commands (“S+S+S+S”) as hardware-specific command data 308B. This command sequence is processed by command processor 130B and transmitted as executable command data 310B to the phoropter 140B.

In some implementations, the system supports additional architectural optimizations. For example, the plugin selection process may be dynamic, leveraging real-time device interrogation or registry-based lookups to identify hardware type and capabilities. The compatibility engine 130A may further include a fallback mechanism that retries command translation with alternate plugins in cases of incompatibility. In other implementations, classification data for each phoropter may be stored centrally and accessed via a networked device manager, enabling centralized governance over plugin deployment and hardware mapping.

As discussed herein, the technique illustrated in FIG. 3 shows how a system supports scalable and robust clinical workflows across diverse hardware platforms. By abstracting clinical intent from device-specific implementation details, the system enables standardized software experiences, ensures compatibility with legacy infrastructure, and facilitates progressive integration of next-generation optometric equipment.

FIGS. 4A and 4B illustrate aspects of an example graphical user interface (GUI) 400 that may be rendered by a client device (e.g., client device 130) during administration of a vision examination. The GUI 400 enables control over clinical workflow steps, presentation of patient-specific vision data, and execution of device commands across a range of phoropter hardware. The GUI 400 may be displayed within a web browser (e.g., as a cloud-based interface), or alternatively, rendered locally on a tablet or laptop, or projected to a clinical display.

As shown in FIG. 4A, the GUI 400 includes a browser toolbar section 402 indicative of a cloud-hosted platform (e.g., “DigitalOptometrics” running on a secure web domain). Below the toolbar is a central data grid region 404 that displays dioptric data for each eye, including values for sphere (S), cylinder (C), axis (A), and add power. The grid may show data from different stages of the workflow, such as autorefractor (AR) readings, lensmeter (LM) readings, and unaided or near visual performance.

A first command region 406 is located to the right of the central data grid and includes GUI elements such as “Clear” and “Fog” buttons. These controls may initiate hardware-level instructions for a connected phoropter (e.g., clearing current lens configuration or fogging one eye). A second command region 404 appears to the left of the data grid and includes an “Export” control, enabling download or transmission of vision test results or patient-specific data to a provider system or electronic health record (EHR) interface. An upper control panel 408 located above the data grid provides directional and binocular targeting options. In some implementations, this includes selection icons for left eye, right eye, or both eyes (e.g., “L,” “R,” “B”), thereby enabling directional control over subsequent phoropter operations.

A lower display region 410 provides a live rendering of an optotype chart to be shown to the patient. The optotype characters (e.g., “RFHZD,” “PTANE,” “FRHOZ”) may vary in size, row, and contrast. This region may be dynamically updated based on workflow progression, patient input, or command feedback from connected hardware. Positioned below the central data grid, this region may serve as a real-time preview of what the patient sees during the examination.

Positioned to the left and right of the optotype display region 410 are additional command zones 412 and 414, respectively. The third command section 412 may include workflow-specific icons that allow the provider to select from various clinical procedures (e.g., vision acuity, chart display, or exam routines). The fourth command section 414 contains arrow-based UI controls to adjust lens values (e.g., sphere or cylinder magnitude and axis orientation) or toggle test parameters. These inputs trigger universal or device-specific commands, depending on the connected hardware and plugin configuration.

FIG. 4B illustrates a workflow-specific interface 416 launched in response to selection of a vision test from the third command section 412. In the example shown, the GUI 416 corresponds to an “Initial Visual Acuity” module. The interface includes a test progress display region 418, which presents a matrix showing completion status for various vision tests across both eyes. This matrix may include fields for unaided results, autorefractor (AR) inputs, and aided values across clinical stages.

The optotype display region 410 remains consistent across FIGS. 4A and 4B, and may be contextually updated to match the current workflow module. Adjacent to this region is a parameter entry panel 420 that includes interactive controls for evaluating test outcomes. These may include buttons for flagging poor visual performance (e.g., “Too Blurry”), selecting the line a patient could read (“Select Line 1”), or specifying the test modality (e.g., unaided, aided, or comparison). In some cases, inputs in this region are used to dynamically advance the workflow, inform command generation, or populate structured health records.

A lower action bar 422 provides navigation controls to progress through or exit from the current test module. Buttons such as “Next,” “Skip Unaided,” and “Cancel” correspond to discrete workflow states and, when engaged, transmit state-change instructions to the underlying application logic (e.g., application 122). The system may also log timestamps, test results, or workflow transitions in response to these controls.

In some implementations, the GUI 400 is dynamically generated based on a standardized clinical workflow definition retrieved from a program plugin (e.g., plugin 114A). This structure allows consistent visual presentation across deployments while supporting variability in test sequence, localization, or device interoperability. The modular nature of the GUI layout supports extensibility to additional modules (e.g., pediatric screening or tele-optometry workflows) and adaptive configuration based on device classification, test context, or patient data.

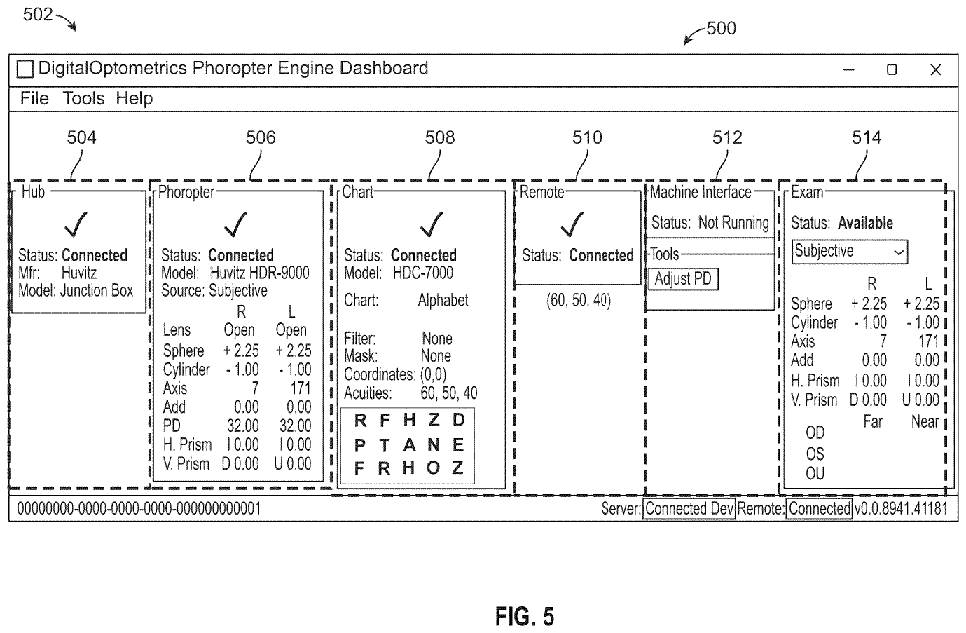

FIG. 5 illustrates an interface 500 configured for use with the system 100 and the GUI 400. The interface 500 operates as a centralized diagnostic and control panel rendered within the GUI 400 and facilitates real-time monitoring and orchestration of vision examination subsystems, including phoropters and eye charts. The interface 500 reflects status information and device-specific diagnostic data across a unified layout, enabling seamless interoperability between hardware components with differing specifications and communication protocols.

In some implementations, the interface 500 integrates with one or more plugins from the plugin library 132, allowing the client device 130 or provider device 120 to interact with heterogeneous devices (e.g., modern digital phoropters, legacy analog systems) using a consistent control interface. Plugins abstract device-specific behavior, allowing the GUI 400 to function as a hardware-agnostic interface layer. The interface 500 can be configured to communicate with controller hardware (e.g., a phoropter hub or communication bridge) based on the particular manufacturer of the phoropter 140 or its eye chart subsystem. In this way, the interface 500 functions as a visual endpoint for confirming device connectivity, synchronizing test results, and validating operational readiness.

The interface 500 includes a visual region 502, which is subdivided into multiple status sections 504 through 514 that each correspond to a system component or communication interface. This visual structure enables a technician 102B or provider 102C to execute clinical workflows without requiring deep familiarity with the operational nuances of individual phoropter models or chart systems. Accordingly, section 504 may display hub-related connection status and manufacturer metadata. Section 506 may present phoropter-specific operational data, including the model name, control mode (e.g., subjective refraction), and measured values for sphere, cylinder, axis, and pupillary distance (PD) across both eyes. Section 508 may reflect the active eye chart device, its model (e.g., HDC-7000), and relevant parameters such as character filtering, masking, and visual acuity coordinate mappings. Section 510 may indicate the connectivity status of the system, including whether the interface is operating in a local, remote, or networked mode. Section 512 may show the operational status of the machine interface, along with adjustable controls (e.g., interpupillary distance) for direct device manipulation. Section 514 may present real-time diagnostic output for the current vision test (e.g., subjective), displaying calculated optical parameters and test states across both left and right eyes. These sections work together to present an integrated view of the diagnostic environment, enabling standardized operation across diverse clinical and hardware contexts.

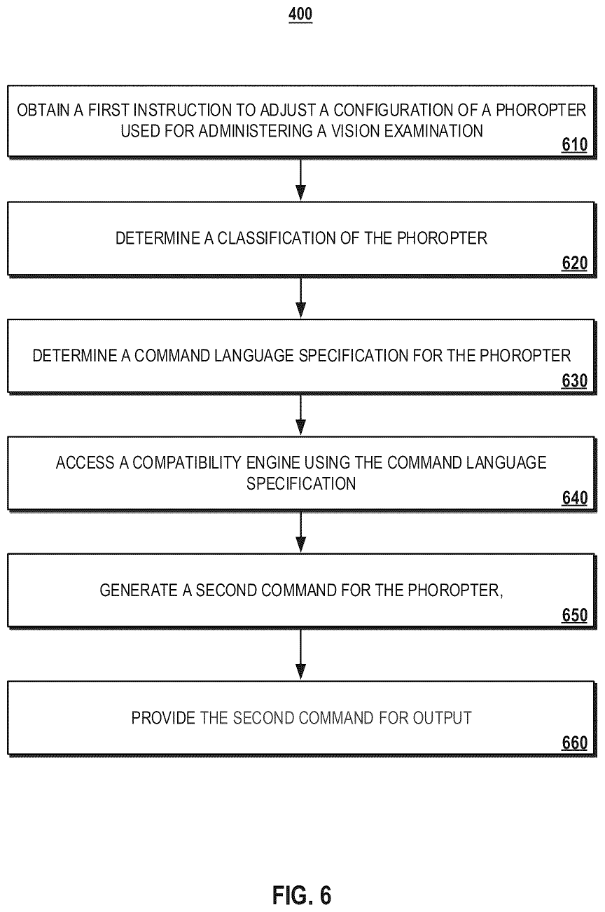

FIG. 6 is a flow chart illustrating an example process 600 for controlling optometric equipment. In general, process 600 includes the operations of obtaining a first instruction to adjust a configuration of a phoropter used for administering a vision examination (610), determining a classification of the phoropter (620), determining a command language specification for the phoropter (630), accessing a compatibility engine using the command language specification (640), generating a second command for the phoropter (650), and providing the second command for output (660).

In more detail, process 600 includes obtaining a first instruction to adjust a configuration of a phoropter (610). For example, a client device 130 receives the first instruction from a provider device 120. In some implementations, the first instruction is obtained from a graphical user interface (GUI), such as GUI 400, executing on the provider device 120. The instruction may be a hardware-independent, universal command corresponding to a specific clinical step in a guided examination workflow, such as a command to perform a Sphere Test or a Jackson Cross Cylinder (JCC) Test.

Process 600 includes determining a classification of the phoropter (620). For example, the compatibility engine 130A of the client device 130 receives data from the connected phoropter 140 that specifies its classification. This information may include the manufacturer, model number, and other hardware identifiers that distinguish it from other types of optometric equipment.

Process 600 includes determining a command language specification for the phoropter (630). For example, based on the classification determined at operation 620, the compatibility engine 130A determines the specific command language required by the phoropter. This specification, which may be defined within a vendor-specific phoropter plugin 114B selected from a plugin library 132, defines the syntax, parameters, and communication protocol for sending valid commands to that particular hardware model.

Process 600 includes accessing a compatibility engine using the command language specification (640). For example, the compatibility engine 130A functions as a hardware abstraction layer. It uses the determined command language specification to access a set of rules for translating the hardware-independent first instruction into a hardware-specific format. In some implementations, the compatibility engine 130A is executed on the client device 130. In other implementations, the compatibility engine 130A is executed on one or more remote computers or servers, and a connection engine enables data communication between the remote computers, the provider device 120, and the client device 130.

Process 600 includes generating a second command for the phoropter (650). For example, the compatibility engine 130A applies the rules defined by the command language specification to the first instruction to generate a second command. This second command is in a format that is compatible with and executable by the specific phoropter being controlled and is configured to adjust a hardware component of the phoropter. For instance, a universal command to add +1.00 sphere may be translated into a verbose command string for one phoropter classification, or into a sequence of single-character step commands for another.

Process 600 includes providing the second command for output (660). For example, the compatibility engine 130A provides the generated second command to a command processor 130B, which transmits control signals representing the second command to the phoropter 140. In some implementations, a connection engine establishes a network connection between the controlling device (e.g., client device 130 or a remote server) and the phoropter prior to receiving the first instruction. The system may also receive, from the phoropter, a connection status indicative of the network connection.

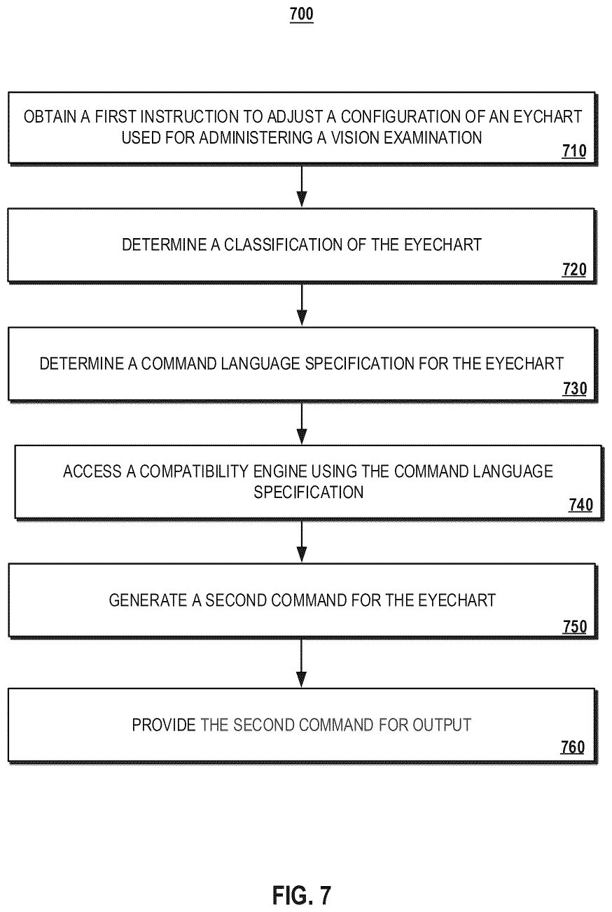

FIG. 7 is a flow chart illustrating an exemplary process 700 for controlling a phoropter using a compatibility engine during a vision examination. In general, process 700, which may be performed by one or more computing devices, includes the operations of obtaining data indicating an instruction to perform an eye chart operation (710), determining a classification of the eye chart (720), determining a command language specification for the eye chart (730), accessing a compatibility engine (740), generating a second command for the eye chart (750), and providing the second command for output (760).

In more detail, process 700 includes obtaining data indicating an instruction to perform an eye chart operation for a vision examination (710). For example, one or more computing devices, such as a client device 130, obtains an instruction that specifies a classification for an eye chart for performing the operation. In some implementations, the instruction is obtained from a graphical user interface (GUI) executing on a computing device, such as a provider device 120. The process may also include establishing a network connection between the one or more computing devices performing the method and the provider device 120 from which the instruction is obtained.

Process 700 further includes determining a first command corresponding to the eye chart operation. For example, the compatibility engine 130A determines a first, universal command based on the instruction. In some implementations, the first command is in a hardware-independent format and is based on identifying a specific clinical step to be performed during the vision examination, such as displaying a Red/Green chart or isolating a specific line of letters.

Process 700 includes determining a classification of the eye chart (720) and determining a command language specification for the eye chart (730). For example, the compatibility engine 130A identifies the model and manufacturer of the connected eye chart display to determine its classification. Based on this classification, the engine determines the specific command language and protocol required to control the eye chart display.

Process 700 includes accessing a compatibility engine (740), which involves selecting a particular plugin corresponding to the first command. For example, the compatibility engine 130A selects, from a library of plugins 132 and based on the eye chart classification, a particular eye chart plugin. The library of plugins identifies a set of eye chart classifications for the vision examination and one or more hardware-specific command rules for each eye chart classification included in the set.

Process 700 includes generating a second command for the eye chart (750). For example, the compatibility engine 130A generates the second command by applying one or more particular hardware-specific command rules specified by the selected eye chart plugin. In some implementations, the second command is in a hardware-specific format that is executable by the eye chart and is determined based on the command language specification associated with the classification for the eye chart. Generating the second command includes generating a command to adjust a hardware component, such as updating the image displayed on a screen.

Process 700 includes providing the second command for output in response to the instruction (760). For example, the command processor 130B receives the second command and provides it for output to the eye chart, causing the displayed chart to change as instructed.

In some implementations, the process 700 further includes receiving, from the device controlling the eye chart, data indicating a connection status.

In some implementations, the set of eye chart classifications in the plugin library includes a first classification specified by the instruction and a second, different classification. The process may further include obtaining second data indicating a second instruction to perform an eye chart operation for a second eye chart having the second classification; determining a third, hardware-independent command; selecting a second particular plugin based on the second classification; generating a fourth, hardware-specific command for the second eye chart by applying rules from the second plugin; and providing the fourth command for output. This allows the system to control different types of eye charts using a consistent workflow.

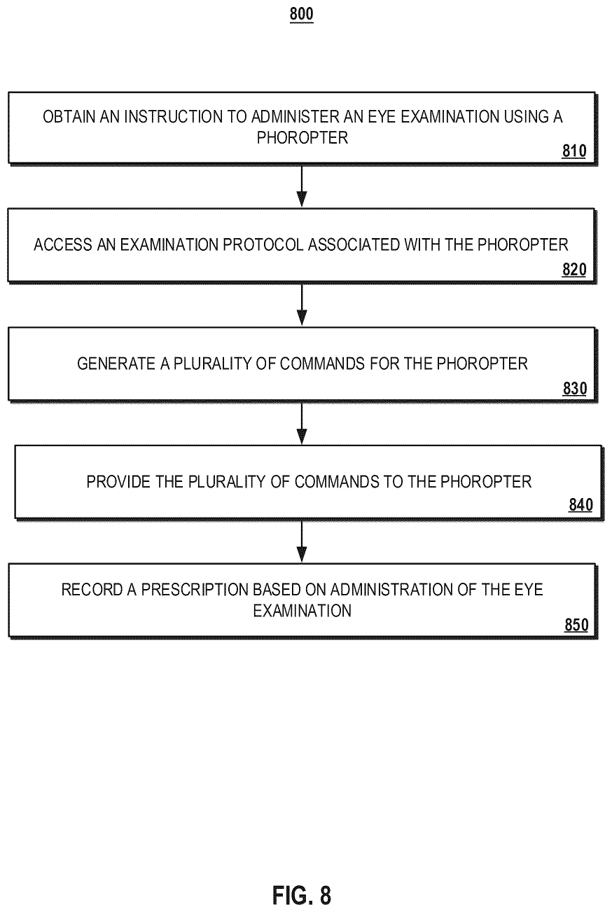

FIG. 8 is a flow chart illustrating an example process 800 for administering a vision examination. In general, process 800 includes the operations of obtaining an instruction to administer a vision examination using a phoropter (810), accessing an examination protocol associated with the phoropter (820), generating a plurality of commands for the phoropter (830), providing the plurality of commands to the phoropter (840), and recording a prescription based on administration of the vision examination (850).

In more detail, process 800 includes obtaining an instruction to administer a vision examination using a phoropter (810). For example, a system obtains a request to begin a guided clinical workflow, which initiates the sequence of automated and rule-based actions for performing a subjective refraction.

Process 800 includes accessing an examination protocol associated with the phoropter (820). For example, the system accesses a predefined protocol that includes a sequence of tests. The protocol may begin with an Initial Visual Acuity test, followed by a Subjective Refraction. The protocol may also include conditional tests to be performed if certain criteria are met, such as a Binocular Balance Test if the visual acuity of both eyes is virtually the same, a Prism Test if the patient has a history of prism in their prescription or complains of double vision, and an Add Power Test if the patient is over 40 years old or complains of difficulty reading.

Process 800 includes generating a plurality of commands for the phoropter (830). For example, the system generates a sequence of commands based on the accessed protocol. For the Initial Visual Acuity test, commands are generated to un-occlude both eyes with no power, then to occlude the left eye to test the right, and then to occlude the right eye to test the left. This sequence may be repeated by generating commands to load Lensometry data and then Auto-Refractor (AR) data into the phoropter. For the Subjective Refraction, a command is generated to load the AR data and occlude the eye not being tested. A command is then generated to fog the tested eye by adding +1.00 to the sphere power; if visual acuity does not improve, a subsequent command adds an additional +0.25 sphere.

As part of the Subjective Refraction, commands are generated for a Jackson Cross Cylinder (JCC) test. If no cylinder is present in the AR data, a command is generated to introduce a −0.50 probing cylinder and add a compensating +0.25 to the sphere. A command is then generated to load the JCC lens for an axis check. An iterative series of commands presents the two JCC positions to the patient; based on the patient's response, a command is generated to change the current axis (e.g., by 10 degrees). This process is repeated, with a rule to halve the adjustment value each time the patient's preference reverses. If the patient initially responds that the options are “about the same,” a command is generated to rotate the JCC by 45 degrees and repeat the check. Once the axis is found, a command is generated to change the JCC to the position for a cylinder power check. A similar iterative process generates commands to adjust the cylinder power (e.g., by −0.25 increments) based on patient preference, while also applying a rule to generate a counteracting command to add +0.25 to the sphere for every −0.50 of cylinder change to maintain the spherical equivalent.

Following the JCC test, the system may generate commands for a final sphere refinement. A command is generated to display a Red/Green visual acuity chart. Based on the patient's response (“Red” or “Green”), a command is generated to add −0.25 or +0.25 to the sphere power, respectively, and the process is repeated until the patient reports they are “about the same.” The entire Subjective Refraction process is then repeated for the other eye.

For conditional tests, specific commands are generated. For the Binocular Balance Test, commands are generated to isolate a single letter on the eye chart and to insert 3 diopters of base down prism on the right eye and 3 diopters of base up prism on the left eye. Based on the patient's response regarding which image is clearer, a command is generated to add +0.25 sphere to the appropriate eye. For the Prism Test, commands are generated to load 12 prism diopters Base In over the right eye and 6 prism diopters Base Down over the left eye, and then to increase or decrease the prism values until the patient reports the targets are aligned. For the Add Power Test, commands are generated to instruct a technician to lower a reading rod, to load a Fused Cross Cylinder lens, and to load an initial add power. Further commands are generated to adjust the sphere power based on whether the patient reports horizontal and vertical lines appear equally clear.

Process 800 includes providing the plurality of commands to the phoropter (840). For example, the commands generated at operation 830 are provided sequentially to the phoropter, causing its hardware components to make the specified adjustments to sphere, cylinder, axis, prisms, and lens states throughout the examination protocol.

Process 800 concludes by recording a prescription based on administration of the vision examination (850). For example, throughout the protocol, the system records various measurements. This includes recording the unaided visual acuity for both eyes, the right eye, and the left eye. It also includes recording the visual acuity with Lensometry data and with Auto-Refractor data for both eyes and each eye individually. After the subjective refraction of an eye is complete, the final sphere, cylinder, and axis are recorded. After the “Binocular Balance” test, the final prescription for far vision is recorded. During the “Prism Test,” the resulting horizontal and vertical prism values are recorded. Finally, during the “Add Power” test, the additional plus power added to the final sphere is recorded as the add power.

Several tests and vision examination functions may be administered using processes 600, 700, and 800, as described above. For example, a “Visual Acuity” test may be conducted to evaluate the patient's visual sharpness under various conditions, including unaided vision, lensometry results, auto-refractor outputs, and subjective refraction outcomes. Visual acuity measurements may be determined for one or both eyes, including OD (right eye), OS (left eye), or OU (both eyes). During a vision examination, appropriate lens values may be loaded into the optometric equipment, and chart displays may be adjusted in real time until the patient's visual acuity reaches a measurable threshold for the eye or eyes under examination.

In some implementations, processes 600, 700, and 800 may be used to enable a standardized and automated framework for guided refraction. These processes may be applied to support accurate, repeatable, and efficient prescription determinations by systematically progressing through defined clinical tests. For instance, a “Sphere” test may be used to determine the initial spherical power and to refine that value after the axis and cylinder have been measured. The “Sphere” test may involve the sequential presentation of two lenses, allowing the provider or patient to compare acuity until no further improvement is observed between lens options.

The “Jackson Cross Cylinder (JCC)” test may be administered to measure both the cylinder axis and cylinder power for a given eye. During the JCC test, a JCC lens included within the optometric equipment may be used to assess the patient's preference between axis orientations. In scenarios where the optometric equipment lacks a physical JCC lens, the compatibility engine 130A may emulate JCC behavior using a predefined lens-switching protocol that mimics JCC functionality through standard spherical and cylindrical lenses.

The “Red/Green” test may be employed to verify whether the sphere power is optimally balanced for the patient. In this test, an eye chart may be displayed with a background split into red and green regions. The patient may be asked which side of the display appears clearer. Based on the patient's response, adjustments may be made to the sphere power in order to achieve perceptual balance between the red and green segments.

The “Binocular Balance” function may be used to equalize visual performance between the left and right eyes, particularly in cases where visual acuity is nearly equivalent in both eyes. During this function, a prism is introduced into the prescription to create image separation. The patient is asked to compare the appearance of the two images, and adjustments are applied until the images appear balanced in brightness, clarity, and alignment.

The “Prism” function may be used to measure and apply prism correction to the patient's prescription to address binocular alignment issues. In this function, prism is incrementally introduced while the patient observes split or duplicated images. The patient indicates when the images appear to align, and based on this feedback, the system determines the appropriate prism correction required to compensate for ocular deviation or imbalance.

The “Add Power” function may be applied to measure the patient's near-vision add power. With this function, the patient may view a near-distance reading chart, and incremental adjustments are made to the add power until reading acuity is optimized. This function supports the accurate determination of multifocal or progressive lens prescriptions by tailoring the near-vision component of the prescription.

The “Final Comparison” function may allow the patient to view and compare their previous prescription against the current prescription derived from the vision examination. This function enables toggling between baseline vision settings (e.g., unaided vision or lensometry data) and subjective refraction results. The system may dynamically adjust the displayed eye chart content to reflect the selected prescription state, enabling visual confirmation of improvements.

The “Smart Cylinder” function may be used to ensure that any adjustment to the cylinder power is compensated with a corresponding adjustment to the sphere power to maintain the spherical equivalent. This compensation helps preserve refractive balance and optimize the prescription outcome. In addition, a “Fog” function may be employed to reduce accommodation by adding a positive diopter value (e.g., +0.50D) to the sphere power, thereby intentionally blurring the image and enabling more accurate subjective refraction.

Implementations of the subject matter and the functional operations described in this specification may be implemented in digital electronic circuitry, in tangibly-embodied computer software or firmware, in computer hardware, including the structures disclosed in this specification and their structural equivalents, or in combinations of one or more of them. Implementations of the subject matter described in this specification may be implemented as one or more computer programs, i.e., one or more modules of computer program instructions encoded on a tangible non-transitory program carrier for execution by, or to control the operation of, data processing apparatus. Alternatively, or in addition, the program instructions may be encoded on an artificially-generated propagated signal, e.g., a machine-generated electrical, optical, or electromagnetic signal, which is generated to encode information for transmission to suitable receiver apparatus for execution by a data processing apparatus. The computer storage medium may be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus may include special purpose logic circuitry, e.g., an FPGA (field programmable gate array) or an ASIC (application-specific integrated circuit). The apparatus may also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) may be written in any form of programming language, including compiled or interpreted languages, or declarative or procedural languages, and it may be deployed in any form, including as a stand-alone program or as a module, component, subroutine, or other unit suitable for use in a computing environment. A computer program may, but need not, correspond to a file in a file system. A program may be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program may be deployed to be executed on one computer or on multiple computers that are located at one site or distributed across multiple sites and interconnected by a communication network.

Computers suitable for the execution of a computer program include, by way of example, may be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer may be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

Implementations of the subject matter described in this specification may be implemented in a computing system that includes a back-end component, e.g., as a data server, or that includes a middleware component, e.g., an application server, or that includes a front-end component, e.g., a client computer having a graphical user interface or a Web browser through which a user may interact with an implementation of the subject matter described in this specification, or any combination of one or more such back-end, middleware, or front-end components. The components of the system may be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular implementations of particular inventions. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems may generally be integrated together in a single software product or packaged into multiple software products.

Devices and techniques for implementing a vision examination using a single controller that is compatible with a plurality of optometric equipment. Particular implementations of the subject matter have been described. Other implementations are within the scope of the following claims. For example, the actions recited in the claims may be performed in a different order and still achieve desirable results. As one example, the processes depicted in the accompanying figures do not necessarily require the particular order shown, or sequential order, to achieve desirable results. In certain implementations, multitasking and parallel processing may be advantageous.

A number of implementations have been described. Nevertheless, it will be understood that various modifications may be made without departing from the spirit and scope of the invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method performed by one or more computing devices, the method comprising:
obtaining data indicating an instruction to perform a phoropter operation for a vision examination, wherein the instruction specifies a classification for a phoropter for performing the phoropter operation;
determining a first command corresponding to the phoropter operation;
selecting, from a library of plugins and based on the classification, a particular plugin corresponding to the first command, wherein the library identifies (i) a set of phoropter classifications for the vision examination and (ii) one or more hardware-specific command rules for each phoropter classification included in the set of phoropter classifications;
generating, by applying one or more particular hardware-specific command rules specified by the particular plugin, a second command for the phoropter, wherein the second command is in a format that is executable by the phoropter; and
providing the second command for output in response to the instruction.

2. The method of claim 1, wherein:
the first command is in a hardware-independent format; and
the first command is based on identifying a specific clinical step to be performed during the vision examination.

3. The method of claim 2, wherein:
the second command is in a hardware-specific format; and
the second command is determined based on a command language specification associated with the classification for the phoropter.

4. The method of claim 1, wherein generating the second command comprises generating a command to adjust a hardware component of the phoropter.

5. The method of claim 1, wherein the instruction is obtained from a graphical user interface (GUI) executing on the computing device.

6. The method of claim 1, further comprising establishing a network connection between the one or more computing devices and the computing device from which the data indicating the instruction is obtained.

7. The method of claim 1, further comprising receiving, from the phoropter, data indicating a connection status.

8. The method of claim 1, wherein:
the library of plugins further includes a plurality of eye chart plugins; and
the method further comprising:
obtaining data indicating an instruction to perform an eye chart operation;
determining a first eye chart command corresponding to the eye chart operation;
selecting, from the plurality of eye chart plugins, a particular eye chart plugin corresponding to a classification of an eye chart;
generating, by applying a hardware-specific command rule from the particular eye chart plugin, a second eye chart command; and
providing the second eye chart command for output to the eye chart.

9. The method of claim 1, wherein:
the set of phoropter classifications includes a first classification specified by the instruction and a second classification different from the first classification,
the method further comprising:
obtaining second data indicating a second instruction to perform a second phoropter operation, wherein the second instruction specifies the second classification for a second phoropter;
determining a third command corresponding to the second phoropter operation;
selecting, from the library of plugins and based on the second classification, a second particular plugin corresponding to the third command;
generating, by applying one or more hardware-specific command rules specified by the second particular plugin, a fourth command for the second phoropter, wherein the fourth command is in a format that is executable by the second phoropter; and
providing the fourth command for output in response to the second instruction.

10. A system comprising:
one or more computing devices; and
one or more storage devices storing instructions that, when executed by the one or more computing devices, causes the one or more computing devices to perform operations comprising:
obtaining data indicating an instruction to perform a phoropter operation for a vision examination, wherein the instruction specifies a classification for a phoropter for performing the phoropter operation;
determining a first command corresponding to the phoropter operation;
selecting, from a library of plugins and based on the classification, a particular plugin corresponding to the first command, wherein the library identifies (i) a set of phoropter classifications for the vision examination and (ii) one or more hardware-specific command rules for each phoropter classification included in the set of phoropter classifications;
generating, by applying one or more particular hardware-specific command rules specified by the particular plugin, a second command for the phoropter, wherein the second command is in a format that is executable by the phoropter; and
providing the second command for output in response to the instruction.

11. The system of claim 10, wherein:
the first command is in a hardware-independent format; and
the first command is based on identifying a specific clinical step to be performed during the vision examination.

12. The system of claim 11, wherein:
the second command is in a hardware-specific format; and
the second command is determined based on a command language specification associated with the classification for the phoropter.

13. The system of claim 10, wherein generating the second command comprises generating a command to adjust a hardware component of the phoropter.

14. The system of claim 10, wherein the instruction is obtained from a graphical user interface (GUI) executing on the computing device.

15. The system of claim 10, wherein the operations further comprise establishing a network connection between the one or more computing devices and the computing device from which the data indicating the instruction is obtained.

16. At least one non-transitory computer-readable storage device storing instructions that, when received by one or more processors, causes the one or more processors to perform operations comprising:
obtaining data indicating an instruction to perform a phoropter operation for a vision examination, wherein the instruction specifies a classification for a phoropter for performing the phoropter operation;
determining a first command corresponding to the phoropter operation;
selecting, from a library of plugins and based on the classification, a particular plugin corresponding to the first command, wherein the library identifies (i) a set of phoropter classifications for the vision examination and (ii) one or more hardware-specific command rules for each phoropter classification included in the set of phoropter classifications;
generating, by applying one or more particular hardware-specific command rules specified by the particular plugin, a second command for the phoropter, wherein the second command is in a format that is executable by the phoropter; and
providing the second command for output in response to the instruction.

17. The non-transitory computer-readable storage device of claim 16, wherein:
the first command is in a hardware-independent format; and
the first command is based on identifying a specific clinical step to be performed during the vision examination.

18. The non-transitory computer-readable storage device of claim 17, wherein:
the second command is in a hardware-specific format; and
the second command is determined based on a command language specification associated with the classification for the phoropter.

19. The non-transitory computer-readable storage device of claim 16, wherein generating the second command comprises generating a command to adjust a hardware component of the phoropter.

20. The non-transitory computer-readable storage device of claim 16, wherein the instruction is obtained from a graphical user interface (GUI) executing on the computing device.
